(19) United States
(12) Patent Application Publication
(10) Pub. No.: US 2025/0095148 A1
(43) Pub. Date: Mar. 20, 2025

(54) METHOD FOR PROVIDING IMAGE ANALYSIS INFORMATION OF BLOOD SAMPLE

(71) Applicant: UIMD INC., Seoul (KR)

(72) Inventor: Young Deuk LEE, Seoul (KR)

(21) Appl. No.: 18/963,878

(22) Filed: Nov. 29, 2024

Related U.S. Application Data
(63) Continuation of application No. PCT/KR2023/006691, filed on May 17, 2023.

(30) Foreign Application Priority Data
Jun. 2, 2022 (KR) 10-2022-0067569

(51) Int. Cl. G06T 7/00

(52) U.S. Cl. CPC G06T 7/0012; G06T 2207/20; G06T 2207/30024

(57) ABSTRACT

A method for testing a blood sample includes capturing, by an image analyzer, a slide sample using an imaging device to obtain a sample image; obtaining, by an image analysis unit of the image analyzer, image analysis data by analyzing the sample image; calling, by a CBC data call unit of the image analyzer, complete blood count (CBC) result data of a complete blood sample that corresponds to the slide sample from a laboratory information system (LIS) server; comparing, by a comparison operation unit of the image analyzer, the CBC result data and the image analysis data based on data correlation; and outputting, by an information output unit of the image analyzer, the CBC result data, the image analysis data, and comparison result.

### CROSS-REFERENCE TO RELATED APPLICATIONS

This application is a Continuation of International Application No. PCT/KR2023/006691 filed on May 17, 2023, which claims priority from Korean Patent Application No. 10-2022-0067569 filed on Jun. 2, 2022. The aforementioned applications are incorporated herein by reference in their entireties.

### BACKGROUND

### 1. Technical Field

The present invention relates to a method for providing image analysis information of a blood sample, and more particularly, to a method for cross-checking and supplementing two-dimensional image analysis information of a blood sample using an image analyzer.

### 2. Description of the Related Art

Complete blood count (CBC) generally refers to a complete blood count that analyzes blood cells, which are tangible components in blood, and there are manual and automated testing methods. Today, laboratories of medical institutions typically use EDTA anticoagulated blood as a sample, and automated testing using an automated blood cell analyzer is common. These automated blood cell analyzers are manufactured by many companies around the world, and the equipment is very diverse.

A CBC test is performed by collecting a small amount of peripheral blood into a vacuum blood collection tube containing EDTA anticoagulant, and the CBC test items include 8 types including white blood cells (WBC), red blood cells (RBC), hemoglobin, hematocrit, three RBC indices, and platelets. In addition, RBC distribution width (RDW) and platelet distribution width (PDW) which indicate changes in the distribution width of red blood cell and platelet sizes, are included in the test items. Other test items include mean corpuscular volume (MCV), mean corpuscular hemoglobin (MCH), and mean corpuscular hemoglobin concentration (MCHC).

An image analyzer (IA) is a device that captures an image of a slide sample smeared with blood and performs classification and differential count of white blood cells (WBC) and red blood cells (RBC), such as WBC Classification and RBC Classification, based on the image analysis results obtained from the captured sample image.

Since the CBC test and the test using the image analyzer mentioned above examine blood samples for different purposes and in different modes, the equipment and interpretation of the results may also be incompatible. Interpreting the results of these different tests and making the resulting diagnosis have been challenging for clinical pathologists or diagnosticians.

In order to provide more accurate and convenient analysis information when clinical pathologists utilize the image analyzers, there is a growing need for research on new methods for providing image analysis information for blood samples.

### SUMMARY

The present invention has been contrived from the above-mentioned necessity, and an object of the present invention is to provide a method for providing image analysis information of a blood sample, which may provide image analysis information to a user, such as a clinical pathologist or a diagnostician, more accurately and more conveniently when examining a slide sample using an image analyzer.

Another object of the present invention is to provide a method for providing image analysis information of a blood sample, which enables a user to confirm and report more accurate analysis results by allowing the blood test results via CBC and image analysis information using an IA to be mutually compared and supplemented.

To achieve the object of the present invention, a method for testing a blood sample may include capturing, by an image analyzer, a slide sample using an imaging device to obtain a sample image; obtaining, by an image analysis unit of the image analyzer, image analysis data by analyzing the sample image; calling, by a complete blood count (CBC) data call unit of the image analyzer, CBC result data of a complete blood sample that corresponds to the slide sample from a laboratory information system (LIS) server; comparing, by a comparison operation unit of the image analyzer, the CBC result data and the image analysis data based on data correlation therebetween; and outputting, by an information output unit of the image analyzer, the CBC result data, the image analysis data, and comparison result from said step of comparing.

The image analysis data may be obtained through image processing by capturing a low-magnification image and a high-magnification image of the slide sample, which has been produced by smear and staining, using the imaging device.

The CBC result data may be obtained by at least one scheme selected from the group consisting of measuring absorbance of a spectral wavelength for the complete blood sample, measuring optical density of the spectral wavelength for the complete blood sample, measuring electrical impedance for the complete blood sample, measuring light scattering for a complete blood sample, and measuring cytochemistry for the complete blood sample.

The data correlation may be evaluated by correlation between each test item of a three-dimensional measurement result of the complete blood sample and a two-dimensional image analysis result of the sample image.

The comparison result may include data match information and/or data mismatch information based on said comparing, and the information output unit may highlight numerical values of each test item that correspond to the data mismatch information.

The comparison result may include data match information and/or data mismatch information based on said comparing, and the method may further include, after said step of outputting, activating, by the information output unit, a data modification window that allows modification of the image analysis data in case of the data mismatch information.

Further, said step of outputting may also include outputting the sample image along with the image analysis data, and outputting comment information based on the output comparison result. Here, the comment information may include a comment regarding the highlighting.

According to an exemplary embodiment of the present invention, when examining a slide sample using an image analyzer, image analysis information may be provided to a user, such as a pathologist or a diagnostician, more accurately and more conveniently.

In addition, by allowing the blood test results through CBC and the image analysis information using the IA to be mutually compared and supplemented, users can confirm and report more accurate analysis results.

### BRIEF DESCRIPTION OF THE DRAWINGS

FIG. 1 is a flowchart sequentially illustrating an exemplary embodiment of a method for providing image analysis information of a blood sample according to the present invention;

FIG. 2 is a configuration diagram illustrating components of an apparatus for executing an exemplary embodiment of the method for providing image analysis information of a blood sample according to the present invention; and

FIG. 3 is a diagram illustrating an example screen displayed by executing an exemplary embodiment of a method for providing image analysis information of a blood sample according to the present invention.

### DETAILED DESCRIPTION

Hereinafter, exemplary embodiments will be described in detail below with reference to the accompanying drawings and contents described in the accompanying drawings, but the present invention is not restricted or limited by the examples.

The exemplary embodiments described below may be subject to various changes. The exemplary embodiments described below are not intended to be limiting of the disclosure, and should be understood to include all modifications, equivalents, or alternatives thereto.

Meanwhile, in describing the present invention, detailed description of associated known function or components will be omitted if they may unnecessarily make the gist of the present invention unclear. Further, terminologies used in this specification are selected for appropriately describing examples of the present invention and may vary depending on an intention of a user or an operator or a practice of a field to which the present invention pertains. Accordingly, definitions of the terminologies need to be described based on the context throughout this specification.

### Method for Providing Image Analysis Information of Blood Sample

An exemplary embodiment of the method for providing image analysis information of a blood sample may be performed so that in providing image analysis data for a sample image to a user by using an image analyzer 10, the image analyzer 10 may communicate with a laboratory information system (LIS) server, CBC result may be called therefrom, the called CBC result data may be compared with the image analysis information, and may output the comparison results.

Hereinafter, the exemplary embodiment will be described in detail with reference to the appended drawings.

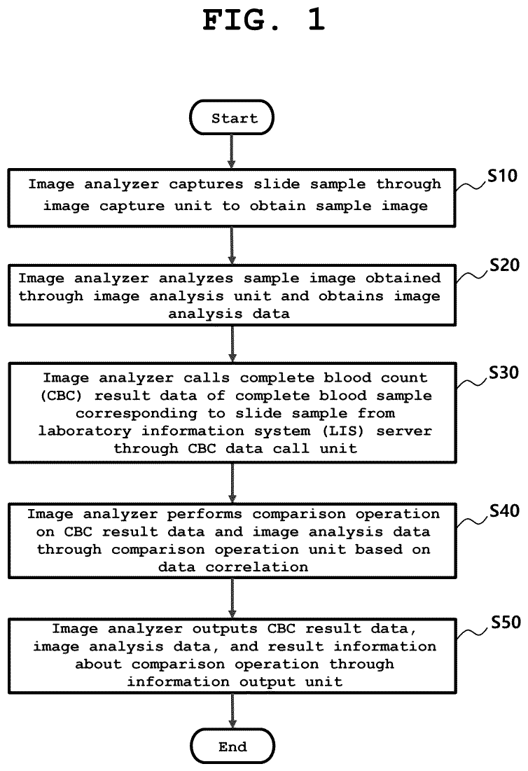

FIG. 1 is a flowchart sequentially illustrating an exemplary embodiment of a method for providing image analysis information of a blood sample according to the present invention. Referring to FIG. 1, in the embodiment, first, an image analyzer 10 may capture an image of a slide sample using an image capture unit (e.g., an imaging device) 100 to obtain a sample image (S10). The image analyzer 10 may perform low-magnification and high-magnification imaging through the image capture unit 100. The slide sample may be prepared for imaging by smearing and staining a small amount of blood on the slide.

Subsequently, the image analyzer 10 may analyze the sample image obtained by the image analysis unit 110 and may obtain image analysis data therefrom (S20). Preceding low-magnification imaging may be performed primarily to select a region of interest (ROI) during subsequent high-magnification imaging. The obtained low-magnification and high-magnification images may undergo appropriate image processing for more accurate analysis.

Further, the image analyzer 10 may call complete blood count (CBC) result data of a complete blood sample corresponding to the slide sample from the laboratory information system (LIS) server 200 through the CBC data call unit 120 (S30). The LIS server 200 may transmit the CBC result data stored in a database 210 to the image analyzer 10. The complete blood count (CBC) result data of the complete blood sample may include the result data from the same patient from a preceding blood test. The CBC result data may be data obtained by at least one of a scheme of measuring absorbance (or optical density) of a spectral wavelength for the complete blood sample, a scheme of measuring electrical impedance for the complete blood sample, a scheme of measuring light scattering for a complete blood sample, or a scheme of measuring cytochemistry for the complete blood sample.

Subsequently, the image analyzer 10 may perform a comparison operation on the CBC result data and image analysis data based on a data correlation through a comparison operation unit 130 (S40). Here, the data correlation may be set as a correlation between each test item of a three-dimensional measurement value of the complete blood sample and a two-dimensional image analysis value of the sample image. For example, the correlation may be set to a higher value for data on the same object (such as red blood cells or white blood cells) in the blood. The correlation may be set to be high for the data where the measurement or analysis values have a causal relationship with each other even if they are not from the same object.

The exemplary embodiment of the method for providing image analysis information of a blood sample may be performed by letting the image analyzer 10 output the CBC result data, image analysis data, and result of the comparison operation through an information output unit 140 (S50). The comparison operation result that is output here may be data match information and/or data mismatch information based on the comparison operation. For example, the data match information may be output as a numerical value, and the data mismatch information may be output so as to provide a visual contrast. In particular, the information output unit 140 may visually output the numerical values of each test item by highlighting the numerical values that are mismatched.

In addition, after the output step S50, in the data mismatch case, the method may further include a step of activating, by the information output unit 140, a data modification window to allow modification of the image analysis data. The data modification window may display grades for degree of the data mismatch, allowing for the user to adjust the grades.

Further, the output step S50 may also include a step of further outputting the sample images, and further outputting comment information based on the comparison operation result that is output. Here, the comment information may be a comment regarding the comparison highlighting.

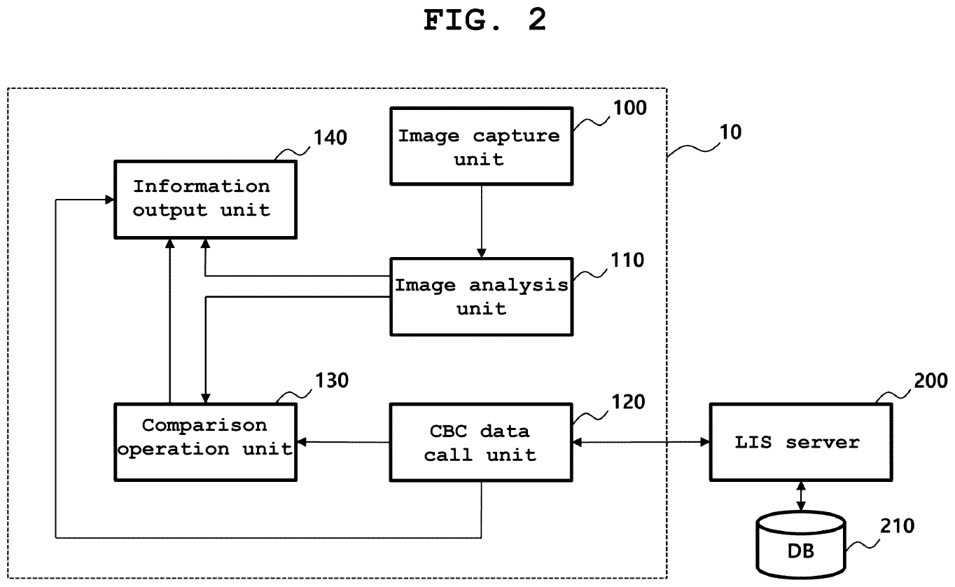

FIG. 2 is a configuration diagram illustrating components of an apparatus for executing an exemplary embodiment of the method for providing image analysis information of a blood sample according to the present invention. The components of the apparatus according to the exemplary embodiment may include an image analyzer 10, an LIS server 200, and a database 210. Further, the image analyzer 10 may include an image capture unit (e.g., an imaging device) 100, an image analysis unit 110, and a CBC data call unit 120.

The image capture unit 100 may include an optical system (e.g., one or more lenses, mirrors, or the like), an image sensor, a drive device, and a drive control unit. In addition, the image analysis unit 110 may be configured as a computing device which may execute storing, processing, and controlling an image processing algorithm required for image analysis.

Further, the CBC data call unit 120 may access a network and be connected to the LIS server 200 through an appropriate communication scheme. The network may be implemented in various types including personal area network (PAN), local area network (LAN), wide area network (WAN), Intranet, Extranet, or the like.

The comparison operation unit 130 may be configured as a computing device capable of executing storing, processing, and controlling a comparison operation program based on a data correlation. Further, the information output unit 140 may be mainly composed of a display device and its peripheral devices and may output information to be visually conveyed to the user.

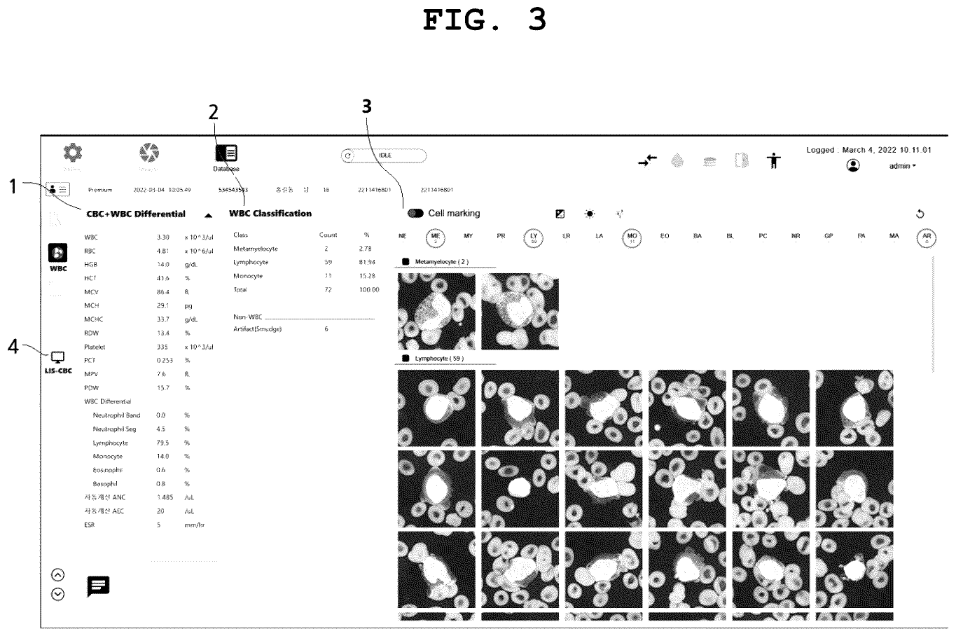

FIG. 3 is a diagram illustrating a first example screen displayed by executing an exemplary embodiment of a method for providing image analysis information of a blood sample according to the present invention. A screen illustrated in FIG. 3 displays a blood cell analysis result of the image analyzer and a blood test result of the complete blood count (CBC) of the same patient on a single screen based on an appropriate UI/UX. Although the image analyzer 10 and the CBC blood test have different testing methods, the image analyzer 10 and the CBC blood test may complement each other and allow comparison and confirmation of the test results. By displaying a CBC blood test result on a first panel 1 and an image analysis on a second panel 2 on the single screen, the user may check and report more accurate analysis results. The CBC blood test results shown in the first panel 1 may be displayed or hidden using an LIS-CBC icon 4.

The complete blood count (CBC) blood test may be performed using a blood analysis device, blood cell image analysis may subsequently be performed on blood with abnormalities, and the results may be compared for reporting the final blood test results. As illustrated in FIG. 3, the second panel 2 may display a test result through blood cell image analysis, and a resulting cell image may be displayed on a third panel 3 based on a user interface (UI) to allow comparison of the test result with the cell image. Misclassification results of the image analyzer 10 may also be checked through the cell image.

### Comparison Operation Based on Data Correlation

The blood cell image analysis result and the blood analysis result (WBC Differential) transmitted to the laboratory information system (LIS) may be automatically compared and analyzed to allow the items with differences to be highlighted in a specific color through 1) test items other than 5-Diff, 2) comparison of result values of the same 5-Diff items, and 3) total count (100 or less).

Here, since the results of items 1), 2), and 3) have different implications, items 1), 2), and 3) may be highlighted in different colors, and since 3) total count (less than 100) is related to the WBC Count and requires additional confirmation, 3) total count (less than 100) may be displayed in another color.

The blood cell image analysis result may contain errors due to various reasons such as poor specimen quality and slide smear condition. When the user reclassifies the misclassified cell image affected by these errors, a comparison highlight may also be displayed for the CBC+WBC Differential result transmitted from the LIS.

Meanwhile, the CBC result data may have, for example, mean corpuscular volume (MCV), mean corpuscular hemoglobin (MCH), and red blood cell distribution width (RDW). The image analysis data from the image analyzer that are related to the CBC result data may include RBC Size Class (Microcyte, Normal, Macrocyte), RBC Chromia Class (Hypochromic, Normal, Hyperchromic), and RBC Size Deviation, respectively.

In an example of a comparison operation using the MCV, the MCH, and the RDW mentioned above, MCV≤80 may mean abundance of microcytes, 81≤MCV≤100 may mean abundance of normals, and MCV≥101 may mean abundance of Macrocytes. Further, MCH≤27 may be regarded as a case of abundant hypochromic, 28≤MCH≤32 may be regarded as a case of abundant normal, and MCH≥33 may be regarded as a case of abundant hyperchromic. In addition, the red blood cell distribution width (RDW) of 11-16% may be regarded as a case where there are abundant normal cells.

Hereinabove, the exemplary embodiments of the present invention have been described with the accompanying drawings, but it can be understood by those skilled in the art that the technical configuration of the present invention can be executed in other detailed forms without changing the technical spirit or requisite features of the present invention. Therefore, it should be appreciated that the aforementioned embodiments are illustrative in any aspects and are not meant to be limiting. In addition, the scope of the present invention is defined by the claims described below rather than the detailed description above. Further, it is to be interpreted that the meaning and scope of the claims and all the changes or modified forms derived from the equivalents thereof fall within the scope of the present invention.

What is claimed is:

1. A method for testing a blood sample, comprising:
capturing, by an image analyzer, a slide sample using an imaging device to obtain a sample image;
obtaining, by an image analysis unit of the image analyzer, image analysis data by analyzing the sample image;
calling, by a complete blood count (CBC) data call unit of the image analyzer, CBC result data of a complete blood sample that corresponds to the slide sample from a laboratory information system (LIS) server;
comparing, by a comparison operation unit of the image analyzer, the CBC result data and the image analysis data based on data correlation therebetween; and
outputting, by an information output unit of the image analyzer, the CBC result data, the image analysis data, and comparison result from said step of comparing.

2. The method of claim 1, wherein the image analysis data is obtained through image processing by capturing a low-magnification image and a high-magnification image of the slide sample, which has been produced by smear and staining, using the imaging device.

3. The method of claim 1, wherein the CBC result data is obtained by at least one of measuring absorbance of a spectral wavelength for the complete blood sample, measuring optical density of the spectral wavelength for the complete blood sample, measuring electrical impedance for the complete blood sample, measuring light scattering for the complete blood sample, or measuring cytochemistry for the complete blood sample.

4. The method of claim 1, wherein the data correlation is evaluated by correlation between each test item of a three-dimensional measurement result of the complete blood sample and a two-dimensional image analysis result of the sample image.

5. The method of claim 4, wherein the comparison result includes data match information and/or data mismatch information based on said comparing, and
wherein the information output unit highlights numerical values of each test item that correspond to the data mismatch information.

6. The method of claim 5, further comprising:
outputting, by the information output unit, comment information based on the comparison result,
wherein the comment information includes a comment regarding said highlighting of the numerical values associated with the data mismatch information.

7. The method of claim 1, wherein the comparison result includes data match information and/or data mismatch information based on said comparing, and
wherein the method further comprises, after said step of outputting:
activating, by the information output unit, a data modification window to allow modification of the image analysis data in case of the data mismatch information.

8. The method of claim 1, further comprising:
outputting, by the information output unit, the sample image along with the image analysis data.
